(19) United States
(12) Patent Application Publication
(10) Pub. No.: US 2011/0104277 A1
(43) Pub. Date: May 5, 2011

(54) OXYGEN BARRIER FILM COATINGS FOR PHARMACEUTICAL DOSAGE FORMS

(76) Inventors: Decheng MA, Wayne, PA (US); Lawrence Rosen, Wayne, PA (US)

(21) Appl. No.: 12/914,144

(22) Filed: Oct. 28, 2010

Related U.S. Application Data
(60) Provisional application No. 61/256,469, filed on Oct. 30, 2009.

(51) Int. Cl. A61K 9/36; A61K 31/40; A61P 9/10; A61P 3/10; A61P 3/04; A61P 9/12; A61P 7/10; A61P 7/02; A61P 3/06; C09D 101/08

(52) U.S. Cl. 424/480; 514/423; 106/163.01; 428/220

(57) ABSTRACT

A film coating which is an oxygen barrier is applied to a pharmaceutical dosage form to protect the dosage form from atmospheric oxygen. The coating comprises a polymeric binder, an anti-tackifying agent, and an optional dissolution enhancing agent, where the polymeric binder is sodium carboxymethylcellulose, the anti-tackifying agent is talc, and the optional dissolution enhancing agent is sucrose, sodium bicarbonate, or a mixture thereof.

### FIELD OF THE INVENTION

This invention relates to barrier coatings that resist penetration by oxygen and that can be applied to pharmaceutical dosage forms, such as tablets, to prevent oxidation of active pharmaceutical ingredients (API's) that are sensitive to oxygen. The invention also relates to the dosage forms and to methods of manufacturing the dosage forms.

### BACKGROUND OF THE INVENTION

Many pharmaceutically active compounds (API's) are sensitive to decomposition caused or accelerated by environmental influences, such as oxygen, humidity, light, heat, and combinations of these. Several approaches have been used to prevent or minimize decomposition caused by atmospheric oxygen. These approaches include the use of anti-oxidants, packaging that excludes air, and various kinds of coatings on dosage forms such as tablets to prevent air from coming into contact with the API. One packaging approach is the use of blister packaging that incorporates an oxygen scavenger in the portion of the blister package which houses the API. A recent review on the prevention of oxidative degradation of API's describes some of these approaches: K. C. Waterman, et al., “Stabilization of Pharmaceuticals to Oxidative Degradation,” Pharmaceutical Development and Technology, 7(1), 1-32 (2002).

Patent publication WO 2004/071403 discloses the use of oxygen barrier coatings or films to prevent oxygen from coming into contact with the API that is encapsulated in the film coating. Not all polymer coatings or films act as barriers to the passage of oxygen. The '403 patent publication discloses the use of the polymer sodium carboxymethylcellulose as a barrier to penetration by oxygen. The invention described herein provides improved coating formulations comprised of sodium carboxymethylcellulose as the base polymer. Furthermore, the coating formulations disclosed herein have advantages compared with the coating formulation disclosed in WO 2004/071403.

### SUMMARY OF THE INVENTION

A protective film coating is provided herein which can be applied to a pharmaceutical dosage form, such as a tablet, to protect it from the effects of atmospheric oxygen, and in particular to prevent degradation that is induced by molecular oxygen. The composition of the film coating comprises (1) a polymeric binder, which is a film-forming polymer, such as sodium carboxymethylcellulose (NaCMC); (2) an anti-tackifying agent, which also acts as a lubricant, as for example talc; and (3) a dissolution enhancing agent, which aids dissolution of the film when the dosage form is administered to a patient. If the film is thin, as for example less than about 55 microns, the film coating can be made comprising the polymeric binder and the anti-tackifying agent without the need for a dissolution enhancing agent. Thinner films (<55 microns in thickness) may optionally include the dissolution enhancing agent, which will generally increase the rate of dissolution of the tablet compared with a tablet that does not include the dissolution enhancing agent. It is generally preferred to include the dissolution enhancing agent even in thinner films to achieve a high rate of dissolution. It has also been discovered that films containing talc as an anti-tackifying agent have better mechanical properties than films that do not contain talc.

### BRIEF DESCRIPTION OF THE DRAWINGS

FIG. 1 is a Stress-Strain curve of (1) a free-standing film comprising sodium carboxymethylcellulose, talc, and NaHCO₃; and (2) a free-standing film comprising OPAGLOS®, a commercial film coating that can be applied to tablets.

### DETAILED DESCRIPTION OF THE INVENTION

The major component in the film coating formulation is the film-forming polymer. A polymer is chosen that can form a continuous barrier film after being applied to the core tablet surface. In general, the film should have low oxygen permeability, good adhesion to the core, and high mechanical strength. Polymers that can be used include sodium carboxymethylcellulose (NaCMC), polyvinyl alcohol (PVA), and copolymers of polyacrylic acid and poly(methyl methacrylate), or mixtures thereof. The preferred polymer is sodium carboxymethylcellulose, which has good mechanical strength, good adhesion, and good barrier properties with respect to oxygen. The film-forming polymer is present in concentrations of 40-80% of the coating by weight, and in other embodiments in the range of 50-80% by weight, or 60-70% by weight of the coating. In preferred embodiments, sodium carboxymethylcellulose is the only film-forming polymer in the protective film coating described herein, excluding small or trace amounts of other polymers that may be present but do not contribute to the properties of the polymeric coating.

The anti-tackifying agents used in the film coating also act as lubricants. Examples of anti-tackifying agents include talc, kaolin, and magnesium stearate. The preferred anti-tackifying agent in the oxygen barrier films is talc, and is preferably micronized talc, as for example micronized talc having D90<10 microns. The anti-tackifying agent is present in concentrations of 10-50% by weight of the coating, and in various embodiments is present in concentrations of 15-35%, 20-30%, 25-35%, or 30%. In various embodiments, talc is the only anti-tackifying agent in the protective film coatings described herein, excluding small or trace amounts of other anti-tackifying agents that may be present but do not contribute to the overall properties of the polymeric coating.

The dissolution enhancing agent is needed for films with thicknesses of about 55 microns or greater to ensure that the film dissolution rate is sufficiently rapid for the API to be released from the film-forming polymer. The dissolution enhancing agent may also optionally be used in thinner films to accelerate the dissolution of the drug. The dissolution enhancing agent is a highly soluble chemical that dissolves rapidly in the aqueous environment, causing the API to be released rapidly by facilitating the disintegration of the dosage form. The dissolution enhancing agents are highly water-soluble chemicals which are selected from non-reducing sugar alcohols, alkali metal salts, carbonate salts and bicarbonate salts. Carbonate salts and bicarbonate salts also can enhance the rate of dissolution of the films in acidic environments by releasing carbon dioxide, resulting in effervescence. Examples of non-reducing sugar alcohols include sucrose, mannitol, xylitol, and trehalose. The preferred dissolution enhancing agents used herein are sucrose (a non-reducing sugar) and sodium bicarbonate, both of which are highly water soluble. Sodium bicarbonate may further accelerate dissolution by forming carbon dioxide gas in acidic environments, thereby causing effervescence. The dissolution enhancing agent is present in concentrations of 0-25%, and in other embodiments 5-25%, 5-20%, or 5-15% of the total amount of solids in the coating.

In preferred embodiments, the film coating contains only (1) sodium carboxymethylcellulose, (2) talc, and (3) NaHCO₃, sucrose, or a mixture thereof, except for small or trace amounts of impurities which do not change the basic and novel characteristics of the film. The films provide protection and stability for API's that are sensitive to chemical degradation induced by molecular oxygen. Examples of such oxygen-sensitive API's include but are not limited to HMG-CoA reductase inhibitors, also known as statins, such as lovastatin, pravastatin, simvastatin, fluvastatin, atorvastatin and rosuvastatin, which may be in lactone or salt forms, and are widely prescribed for reducing LDL-cholesterol (the “bad” cholesterol) and for treating lipid disorders, such as dyslipidemia, hyperlipidemia, and hypercholesterolemia. Amorphous atorvastatin, and particularly amorphous atorvastatin calcium, is more sensitive to air oxidation than the crystalline form of atorvastatin calcium, which is marketed under various names including LIPITOR®. The film coating disclosed herein is particularly useful with core tablets containing amorphous atorvastatin calcium.

The core tablet to be coated may optionally include one or more active agents in a therapeutically effective amount in addition to the oxygen sensitive API. The amount of additional active agent or agents which may be present will depend on the therapeutically desirable amount of additional active agent or agents and therapeutically desirable amount of the oxygen sensitive API per dosage unit, maximum feasible dosage unit size, and the physical and chemical properties of the optional additional active agent or agents.

As an example, when the oxygen sensitive API is atorvastatin (particularly amorphous atorvastatin calcium), exemplary additional active agents in the core tablet include anti-atherosclerotic agents; anti-diabetes agents; anti-obesity agents; anti-hypertensive agents; agents used for the treatment of metabolic syndrome; lipid modifying agents; agents that have both lipid-modifying effects and other pharmaceutical activities; cholesterol absorption inhibitors such as ezetimibe (ZETIA®) which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. RE37721 and 5,846,966; HDL-raising agents such as cholesterol ester transfer protein (CETP) inhibitors, for example anacetrapib, also known as MK-0859 and having the chemical name (4S,5R)-5-[3,5-bis(trifluoromethyl)phenyl]-3-{[4′-fluoro-2′-methoxy-5′-(1-methylethyl)-4-(trifluoromethyl)[1,1′-biphenyl]-2-yl]methyl}-4-methyl-2-oxazolidinone (described in WO2006/014357, Merck & Co. Inc.) and dalcetrapib, also known as JTT-705 (Japan Tobacco Company, Roche); reverse cholesterol transport enhancers; other HMG-CoA synthase inhibitors; diuretics, for example hydrochlorothiazide; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; niacin in immediate and controlled release form, optionally with an anti-flushing agent such as a DP antagonist such as laropiprant, and particularly the product known as TREDAPTIVE® which contains controlled-release niacin in one layer and laropiprant in the other layer of a bilayer tablet; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate and gemfibrozil; PPAR dual α/γ agonists such as muraglitazar; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan and losartan with hydrochlorothiazide; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all subtypes of this receptor, such as LXRα and LXRβ; bisphosphonate compounds such as alendronate sodium; cyclooxygenase-2 inhibitors such as rofecoxib and celecoxib; sibutramine; orlistat; topiramate; naltrexone; bupriopion; phentermine; phentermine/topiramate combination (QNEXA®); NPY5 antagonists; Acetyl-CoA Carboxylase-1 and -2 (ACC) inhibitors; MCH1R antagonists; CB1 antagonists/inverse agonists such as those described in WO03/077847 and WO05/000809; DPP-4 (dipeptidylpeptidase-4) inhibitors such as sitagliptin (JANUVIA®) and vildagliptin (GALVUS®); sulfonylureas such as chlorpropamide, tolazamide, glyburide, glipizide, and glimepiride; biguanides, such as metformin; alpha-glucosidase inhibitors such as acarbose and miglitol; meglitinides such as repaglinide; glucagon-receptor agonists; and glucokinase activators. This list of active agents is illustrative and not intended to be exhaustive.

Exemplary core tablets include monolithic (i.e., single layer), bi-layer and multi-layer tablets. In one embodiment, a monolithic tablet comprises atorvastatin optionally admixed with excipients and one or more additional active agents then compressed into a tablet, for example wherein atorvastatin is admixed in a composition with a cholesterol absorption inhibitor (e.g. ezetimibe), a high density lipoprotein (HDL) raising agent (e.g., niacin in immediate or extended release form), a CETP inhibitor (e.g. anacetrapib), or an anti-diabetic agent (e.g. sitagliptin). Alternatively, a multi-layer tablet, particularly a bilayer tablet, can be employed. In bilayer and multi-layer embodiments, each tablet layer may contain one or more pharmaceutically active agents, selected independently of the agents present in any other tablet layer. In one embodiment, one layer of a bi-layer tablet can be comprised of the atorvastatin and the second layer can be comprised of a different additional active agent or agents. For example, a film-coated tablet of this invention comprising atorvastatin is present in one layer and the second layer is comprised of a cholesterol absorption inhibitor such as ezetimibe, niacin (immediate or extended release), a CETP-inhibitor such as anacetrapib, or an anti-diabetic agent such as sitagliptin. Another exemplary bi-layer tablet comprises extended-release niacin in one layer, and atorvastatin admixed with a DP-antagonist, particularly laropiprant, in the second layer. The type of solid dosage form can be determined by one of skill in the art based upon a variety of considerations such as, for example, the compatibility or desired release profile of the active agents and the desired therapeutic effect.

The process for making and applying the coating to the core comprises the step of first dispersing the components of the coating into a solvent at room temperature to yield a suspension. Solvents that can be used for the coating dispersion include water and various water-alcohol mixtures. Water is the preferred solvent and is generally the solvent that is used. The suspension is atomized and sprayed onto the core tablets using standard pan coating equipment. The film coat solidifies and adheres to the core tablet after drying. The solvent, preferably water, is added in concentrations of 92-96%, and preferably 92.5-94.5% of the total amount of the coating solution. The thickness of the film that is coated onto the core is in the range of 30-120 microns, and preferably in the range of 30-55 microns. As stated previously, the preferred coatings, which are less than 55 microns in thickness, do not need to have a dissolution enhancing agent in the compositions, although even these coatings generally include a dissolution enhancing agent as part of the composition.

The preferred composition of the coating formulation contains NaCMC (60-70% by weight of the solids) as the coating polymer, talc (20-30%) as the anti-tackifying agent, and sodium bicarbonate (0-10%) or sucrose (0-20%) as the dissolution enhancing agent. The resulting film coating has the same % composition of solids as the coating formulation. The film coating that results from using this formulation can be used as an oxygen barrier film coating for solid dosage forms, preferably tablets. This film coating provides good protection for API's which are sensitive to degradation by molecular oxygen. The film coating formulation that is used in making the coatings is compatible with acidic, basic, primary amine, and salt forms of API's and with a broad range of common pharmaceutical excipients that are used in the core tablet formulations.

### EXAMPLES

The following examples are provided to illustrate the invention and are not to be construed as limiting the scope of the invention in any way.

### ABBREVIATIONS

The following abbreviations and expressions have the meanings indicated herein, unless stated otherwise in the specification:

FCT—film coated tablet
QC method—Dissolution in pH 6.8 medium
RH—relative humidity
SGF—simulated gastric fluid, having pH=1.8
w/w—weight/weight

Examples 1 and 2 illustrate coating compositions that contain sodium carboxymethylcellulose, talc, and water along with sodium bicarbonate in Example 1 and sucrose in Example 2. The water is present as a carrier for the dissolved and suspended solids, and is evaporated after the compositions are applied to the cores of the tablets.

### Example 1

### Coating Composition Containing NaHCO₃

| COMPONENT | % IN SOLID FILM | % IN SOLUTION |
|---|---|---|
| NaCMC [Aqualon | 60 | 4.000 |
| CMC, 7LFPH] | | |
| Talc [LoMicron 5, | 30 | 2.00 |
| d90 < 10 μm] | | |
| NaHCO₃ [Compendial] | 10 | 0.67 |
| Water [USP] | | 93.33 |
| Total | 100 | 100 |
| Solid concentration | | 6.7% |
| (w/w) | | |

Sodium carboxymethycellulose (4 g) having a viscosity of 25-50 cp was dissolved in water (61.6 g) while mixing. Then, while still mixing, a dispersion of excipient concentrate was added. The excipient concentrate was prepared by dispersing NaHCO₃ (0.67 g) and talc (2.0 g) in water (31.7 g) by stirring. The resulting dispersion was atomized and sprayed onto the core tablets using a Vector LCDS3 spray coating apparatus to obtain the desired 4-8% by weight with respect to the core mass. Typical coating parameters are listed in the table below:

PARAMETER (VECTOR LDCS 3)

| | |
|---|---|
| Minimal Weight Gain (mg/mm2) | 0.08 |
| Solution Concentration (% solid) | 6.7 |
| Solution viscosity (cp) | 400 |
| Batch Size (g) | 100 |
| Pan Size (in, fully perforated) | 8.5 |
| Pan Speed (rpm) | 20 |
| Exhaust Temperature (° C.) | 47 |
| Inlet Air Temp (° C.) | 65-75 |
| Inlet Air Flow (cfm) | 35-40 |
| Nozzle Cap | 134255-45°-SS |
| Spray Rate (g/min) | 1.2-2.0 |
| Atomization Pressure (psi) | 20 |
| Exhaust RH (%) | <20 |
| Coating Time (min) | 60-80 |

### Example 2

### Coating Composition Containing Sucrose

| COMPONENT | % IN SOLID FILM | % IN SOLUTION |
|---|---|---|
| NaCMC [Aqualon | 60 | 4.000 |
| CMC, 7LFPH] | | |
| Talc [LoMicron 5, | 20 | 1.33 |
| d90 < 10 μm] | | |
| Sucrose [Compendial] | 20 | 1.33 |
| Water [USP] | | 93.34 |
| Total | 100 | 100 |
| Solid conc. (w/w) | | 6.7% |

Sodium carboxymethycellulose (4 g) having a viscosity of 25-50 cp was dissolved in water (61.6 g) while mixing. Then, while still mixing, a dispersion of excipient concentrate was added. The excipient concentrate was prepared by dispersing sucrose (1.33 g) and talc (1.33 g) in water (31.7 g) by stirring. The resulting dispersion was atomized and sprayed onto the core tablets to obtain the desired 4-8% by weight with respect to the core mass. Typical coating parameters are listed below:

PARAMETER (VECTOR LDCS 3)

| | |
|---|---|
| Minimal Weight Gain (mg/mm2) | 0.08 |
| Solution Concentration (% solid) | 6.7 |
| Solution viscosity (cp) | 400 |
| Batch Size (g) | 100 |
| Pan Size (in, fully perforated) | 8.5 |
| Pan Speed (rpm) | 20 |
| Exhaust Temperature (° C.) | 47 |
| Inlet Air Temp (° C.) | 65-75 |
| Inlet Air Flow (cfm) | 35-40 |
| Nozzle Cap | 134255-45°-SS |
| Spray Rate (g/min) | 1.2-2.0 |
| Atomization Pressure (psi) | 20 |
| Exhaust RH (%) | <20 |
| Coating Time (min) | 60-80 |

### Examples 3-5

Tables of data are provided showing the % degradation of the API in the tablets resulting from the action of molecular oxygen (Example 3), degradation of the API in the tablets resulting from lactonization and other reactions brought about by moisture and possibly acid (Example 4), and dissolution data for the tablets as initially made in simulated gastric fluid (SGF) at pH 1.8 and using the QC test method at pH 6.8.

The QC dissolution method uses USP Apparatus II with a paddle speed of 75 rpm and a bath temperature of 37° C. The dissolution is performed in 900 mL of 50 mM phosphate at pH 6.8. Samples are removed from the dissolution vessels at time intervals of 10, 15, 20, 30, 45, and 60 minutes. Dissolution samples are analyzed with a HPLC method using a Phenomenex Onyx Monolithic C18 100×4.6 mm, 10 um particle size column. The mobile phase consists of a 55%/45% (v/v) solution of 0.1% acetic acid: acetonitrile. The flow rate is 5.0 mL/min, the injection volume is 10 microliters, and the column temperature is 45° C. Detection is by UV at 244 nm. The retention time for the API peak is approximately 1.2 min.

The SGF dissolution method uses USP Apparatus II with a paddle speed of 100 rpm and a bath temperature of 37° C. The dissolution is performed in 250 mL of SGF, which contains 1.4 mg/mL hydrochloric acid and 2 mg/mL sodium chloride and has pH of 1.8. Samples are removed from the dissolution vessels at time intervals of 10 and 30 minutes. Dissolution samples are analyzed with a HPLC method using a Phenomenex Onyx Monolithic C18 100×4.6 mm, 10 um particle size column. The mobile phase consists of a 55%/45% (v/v) solution of 0.1% acetic acid: acetonitrile. The flow rate is 5.0 mL/min, the injection volume is 10 microliters, and the column temperature is 45° C. Detection is by UV at 244 nm. The retention time for the API peak is approximately 1.2 min.

The assay and degradate method for the API uses a reversed phase HPLC method with a Symmetry Shield RP18 (150×4.6 mm, 3.5 μm particle size) column. The mobile phase consists of 20 mM Acetate pH 4.0 and acetonitrile. The flow rate is 0.9 mL/min, the injection volume is 12 μL and the column temperature is 25° C. The autosampler tray temperature is set at 10° C. Detection is by UV at 244 nm. The retention time for the active peak is 15.7 minutes.

The data are presented for uncoated tablets (controls) and for tablets coated with compositions having the following components at the following weight %:

| | NaCMC, low | Talc, | | |
|---|---|---|---|---|
| | viscosity, | [LoMicron 5, | | |
| | [Aqualon | micronized | | |
| Component | CMC, 7LFPH] | (d90 < 10 μm)] | Sucrose | NaHCO₃ |
| NaCMC-Talc | 70 | 30 | 0 | 0 |
| NaCMC-Talc- | 60 | 30 | 10 | 0 |
| 10% Sucrose | | | | |
| NaCMC-Talc- | 65 | 30 | 0 | 5 |
| 5% NaHCO₃ | | | | |
| NaCMC-Talc- | 60 | 30 | 0 | 10 |
| 10% NaHCO₃ | | | | |

The cores of all of the tablets have the composition shown below:

| Composition | mg/tablet | Component |
|---|---|---|
| 13.85% | 41.55 | Amorphous atorvastatin calcium (004G) |
| 47.50% | 142.50 | Cellulose, Microcrystalline, Compendial [Avicel PH-102] |
| 28.20% | 84.60 | Lactose, Anhydrous, Compendial [Direct Tabletting] |
| 3.00% | 9.00 | Hydroxypropyl Cellulose, Compendial [Klucel EXF] |
| 3.00% | 9.00 | Croscarmellose Sodium, Compendial |
| 2.00% | 6.00 | Sodium Bicarbonate, Compendial, Powder (PhEur - Sodium |
| | | Hydrogen Carbonate) |
| 1.00% | 3.00 | Sodium Lauryl Sulfate, Compendial, [Texapon K 12 P PH] |
| 0.50% | 1.50 | Magnesium Stearate, Compendial [Non-Bovine], Intragranular |
| 1.00% | 3.00 | Magnesium Stearate, Compendial [Non-Bovine], Extragranular |

The cores were made by dry granulation followed by compression into tablets. The materials were purchased from commercial sources or were made using published methods. The API and excipients, except magnesium stearate, were blended in a PK blender at 25 rpm for 10 minutes. Magnesium stearate (0.5% of final composition) was then added and blended for another 5 minutes at 25 rpm. The mixture was then transferred to a WP120×40 mm roller compactor and granulated into dry ribbons at 60 bar roll pressure. The granulation was then milled at 100 rpm through a 2.0 and 0.8 mm Conidur screen on the roller compactor. The granules were finally mixed with magnesium stearate (1% of final composition) and lubricated in a PK blender for 5 minutes at 25 rpm. The final formulation was compressed into tablets in a Korsh XL100 rotary press with a 12/32″ round standard concave tooling at 9.5 kN compression force. The tablets were then tested without being coated or were coated with the coating solution and then dried.

### Example 3

### Chemical Stability Test Data for Degradation Resulting from Molecular Oxygen—% Degradation of API after 2 and 4 weeks

| | | | 2 weeks | | | 4 weeks |
|---|---|---|---|---|---|---|
| Film | Film | Coating | | 40° C./75% | 60° C./Amb | 40° C./75% |
| Coated | Thickness | Weight | 30° C./65% | RH Closed | Closed | RH Closed |
| Tablets | (μm) | Gain | Open | w/desiccant | w/desiccant | w/desiccant |
| Uncoated | 0 | 0 | 0.23 | 0.42 | 1.10 | 0.76 |
| Core- | | | | | | |
| Control | | | | | | |
| NaCMC- | 29 | 3.6% | 0.07 | 0.04 | 0.09 | 0.32 |
| Talc | | | | | | |
| NaCMC- | 56 | 5.8% | 0.12 | 0.11 | 0.04 | 0.12 |
| Talc | | | | | | |
| NaCMC- | 56 | 5.8% | 0.03 | 0.09 | 0.04 | 0.11 |
| Talc-10% | | | | | | |
| Sucrose | | | | | | |
| NaCMC- | 56 | 5.8% | 0.11 | 0.05 | 0.03 | 0.17 |
| Talc-5% | | | | | | |
| NaHCO₃ | | | | | | |
| NaCMC- | 56 | 5.8% | 0 | 0.05 | 0 | 0.05 |
| Talc-10% | | | | | | |
| NaHCO₃ | | | | | | |

### Example 4

### Chemical Stability Test Data for Degradation Resulting from Exposure to Moisture and Acidity—% Degradation of API after 2 and 4 weeks

| | | | 2 weeks | | | 4 weeks |
|---|---|---|---|---|---|---|
| Film | Film | Coating | | 40° C./75% | 60° C./Amb | 40° C./75% |
| Coated | Thickness | Weight | 30° C./65% | RH - Closed | Closed | RH Closed |
| Tablets | (μm) | Gain | RH - Open | w/desiccant | w/desiccant | w/desiccant |
| Uncoated | 0 | 0 | 0.13 | 0.03 | 0.15 | 0.04 |
| Core- | | | | | | |
| Control | | | | | | |
| NaCMC- | 29 | 3.6% | 0.14 | 0.04 | 0.14 | 0.03 |
| Talc | | | | | | |
| NaCMC- | 56 | 5.8% | 0.15 | 0.03 | 0.15 | 0.04 |
| Talc | | | | | | |
| NaCMC- | 56 | 5.8% | 0.14 | 0.04 | 0.24 | 0.05 |
| Talc-10% | | | | | | |
| Sucrose | | | | | | |
| NaCMC- | 56 | 5.8% | 0.12 | 0.03 | 0.24 | 0.07 |
| Talc-5% | | | | | | |
| NaHCO₃ | | | | | | |
| NaCMC- | 56 | 5.8% | 0.12 | 0.06 | 0.25 | 0.08 |
| Talc-10% | | | | | | |
| NaHCO₃ | | | | | | |

### Example 5

### Initial Tablet Dissolution Data

| | | | Dissolution (% released) | | |
|---|---|---|---|---|---|
| Film | Film | Coating | SGF (Biorelevant | | pH 6.8 |
| Coated | Thickness | Weight | method) | | (QC method) |
| Tablets | (μm) | Gain | 15 min | 30 min | 10 min |
| Uncoated | 0 | 0 | 14 | 17 | 48 |
| Core- | | | | | |
| Control | | | | | |
| NaCMC- | 29 | 3.6% | 10 | 17 | 45 |
| Talc | | | | | |
| NaCMC- | 56 | 5.8% | 3 | 15 | 35 |
| Talc | | | | | |
| NaCMC- | 56 | 5.8% | 11 | 17 | 45 |
| Talc-10% | | | | | |
| Sucrose | | | | | |
| NaCMC- | 56 | 5.8% | 10 | 16 | 46 |
| Talc-5% | | | | | |
| NaHCO₃ | | | | | |
| NaCMC- | 56 | 5.8% | 11 | 17 | 48 |
| Talc-10% | | | | | |
| NaHCO3 | | | | | |

### Example 6

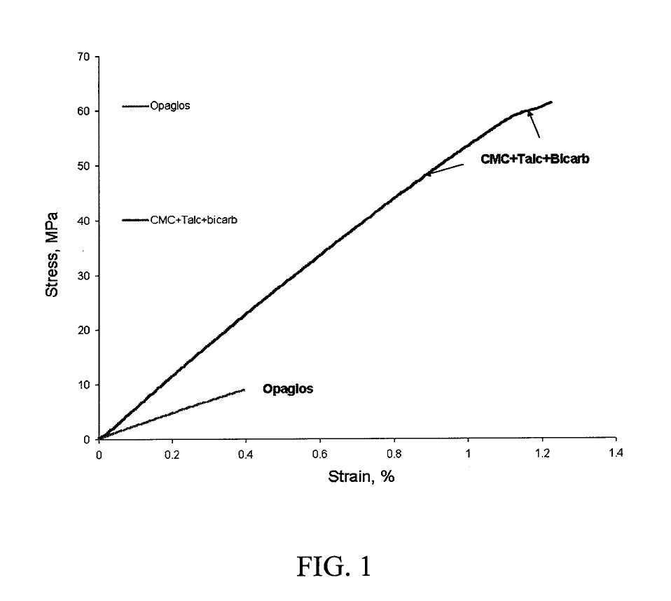

A solution of 60% (w/w) NaCMC, 30% talc, and 10% NaHCO₃ in water was cast into a free-standing film at room temperature or below. A free-standing film of the Opaglos was made by the same method as the NaCMC/talc/NaHCO₃ film. The films were equilibrated in air at room temperature and 15% relative humidity for 24 hours. Tests measuring stress at increasing strain were carried out using a Texture Analyzer, and were carried out until the films broke. The stress-strain plots were obtained using the software that came with the instrument. The plots are shown in FIG. 1. The plot for the NaCMC/talc/NaHCO₃ film, which is labeled CMC+Talc+Bicarb in FIG. 1 has a nearly linear slope almost to the inflection point at 1.2% strain. The Opaglos film has a much shorter, less steep, linear plot compared with the NaCMC/talc/NaHCO₃ film. The NaCMC/talc/NaHCO₃ film is stronger and tougher based on this test.

### Example 7

Pharmacokinetic parameters were measured for a fast dissolving tablet formulation of amorphous calcium atorvastatin and a formulation of a second API that would be used in combination with atorvastatin either in a fixed dose combination or by co-administration. The formulations were tested using panels of 30 subjects each. The following pharmacokinetic (PK) measurements were carried out. The procedures are further summarized below the table:

GMR (90% CI)

| | | |
|---|---|---|
| PK Parameters | (1)/(3) | (2)/(3) |

Second API

| | | |
|---|---|---|
| AUC_{0-∞} | 1.01 (0.99, 1.03) | 1.03 (1.01, 1.05) |
| Cmax | 1.00 (0.93, 1.08) | 0.99 (0.92, 1.06) |

Atorvastatin

| | | |
|---|---|---|
| AUC_{0-∞} | 1.00 (0.91, 1.09) | 1.00 (0.91, 1.09) |
| Cmax | 1.12 (0.93, 1.35) | 0.98 (0.82, 1.18) |

Treatment (1): Administration of the bilayer tablets of Atorvastatin (80 mg) and second API (100 mg), uncoated
Treatment (2): Administration of the bilayer tablets of atorvastatin (80 mg) and second API (100 mg), film coated with NaCMC/talc/NaHCO3 oxygen barrier film coating
Treatment (3): Co-administration of commercial atorvastatin (80 mg) and second API (100 mg) tablets, coated with non-protective, tastemasking film coating (e.g. Opadry ®), based on the packaging label

(1) The amorphous atorvastatin formulation and the second API were made into a bilayer tablet which was administered as a fixed dose combination without a protective coating.

(2) The amorphous atorvastatin formulation and the second API were made into a bilayer tablet which was coated with the NaCMC/talc/NaHCO₃ oxygen barrier film coating. The tablet was administered as a fixed dose combination.

(3) The commercial atorvastatin tablets and the second API tablets, which were not coated with a protective layer, were co-administered.

Data specific to atorvastatin follow:

Pharmokinetic measurements (AUC_{0-∞} and Cₘₐₓ) were measured as described above. The measurements for the first atorvastatin formulations were compared by calculating the ratios of the AUC_{0-∞} and Cₘₐₓ for the atorvastatin in the fixed dose combination (1) above and the AUC_{0-∞} and Cₘₐₓ for the atorvastatin that was coadministered in (3) above. The ratios of the lower and upper limits of the 90% confidence interval for each measurement were also computed. The ratio of AUC_{0-∞} including the ratios of the confidence intervals was 1.00 (0.91, 1.09). The ratio of Cₘₐₓ including the ratios of the confidence intervals was 1.12 (0.93, 1.35).

The measurements for the second atorvastatin formulations (Nos. 2) were compared by calculating the ratios of the AUC_{0-∞} and Cₘₐₓ for the atorvastatin in the fixed dose combination (2) above and the AUC_{0-∞} and Cₘₐₓ for the atorvastatin that was coadministered in (3) above. The ratios of the lower and upper limits of the 90% confidence interval for each measurement were also computed. The ratio of AUC_{0-∞} including the ratios of the confidence intervals was 1.00 (0.91, 1.09). The ratio of Cₘₐₓ including the ratios of the confidence intervals was 0.98 (0.82, 1.18).

By comparison of PK data from treatment (1) and (2), it appears that the oxygen barrier film coat (OBFC) has no effect on the atorvastatin AUC. The Cₘₐₓ is lower for the OBFC tablets, indicating the dissolution slowing effect of the coating. However, the confidence intervals are still in the BE acceptable range, which is (0.80, 1.25).

While the invention has been described and illustrated with reference to certain particular embodiments thereof, those skilled in the art will appreciate that various adaptations, changes, modifications, substitutions, deletions, or additions of procedures, protocols and compositions may be made without departing from the spirit and scope of the invention. Other embodiments of the invention will be apparent to those skilled in the art from consideration of the specification and examples disclosed herein. It is intended that the specification and examples be considered as exemplary only, and that the invention be defined by the scope of the claims which follow, and that such claims be interpreted as broadly as is reasonable.

What is claimed is:

1. A protective film coating for a pharmaceutical dosage form, wherein the coating comprises a polymeric binder and an anti-tackifying agent, wherein the polymeric binder is sodium carboxymethylcellulose and the anti-tackifying agent is talc.

2. The protective film coating of claim 1, wherein the thickness of the coating is in the range 30-55 microns.

3. The protective film coating of claim 1, wherein the coating comprises sodium carboxymethylcellulose, talc, and a dissolution enhancing agent.

4. The protective film coating of claim 3, wherein the thickness of the coating is in the range of 30-120 microns.

5. The protective film coating of claim 3, wherein the thickness of the coating is in the range of 30-55 microns.

6. The protective film coating of claim 3, wherein the coating comprises sodium carboxymethylcellulose, talc, and a dissolution enhancing agent selected from sucrose and sodium bicarbonate, or a mixture thereof.

7. The protective film coating of claim 6, wherein the dissolution enhancing agent is sucrose.

8. The protective film coating of claim 6, wherein the dissolution enhancing agent is sodium bicarbonate.

9. The protective film coating of claim 1, wherein the coating comprises 50-80% (w/w) of sodium carboxymethylcellulose and 15-35% (w/w) of talc.

10. The protective film coating of claim 1, wherein the coating comprises 50-80% (w/w) of sodium carboxymethylcellulose, 15-35% (w/w) of talc, and 0-25% (w/w) of an optional dissolution enhancing agent.

11. The protective film coating of claim 3, wherein the coating comprises 50-80% (w/w) of sodium carboxymethylcellulose, 15-35% (w/w) of talc, and 5-25% (w/w) of the dissolution enhancing agent.

12. The protective film coating of claim 11, wherein the dissolution enhancing agent is sodium bicarbonate.

13. The protective film coating of claim 11, wherein the dissolution enhancing agent is sucrose.

14. The protective film coating of claim 3, wherein the coating comprises 60-70% (w/w) of sodium carboxymethylcellulose, 20-30% (w/w) of talc, and 5-20% (w/w) of the dissolution enhancing agent.

15. The protective film coating of claim 14, wherein the dissolution enhancing agent is sodium bicarbonate.

16. The protective film coating of claim 14, wherein the dissolution enhancing agent is sucrose.

17. The protective film coating of claim 1, wherein the coating consists essentially of sodium carboxymethylcellulose and talc.

18. The protective film coating of claim 17, wherein the coating thickness is in the range of 30-55 microns.

19. The protective film coating of claim 6, wherein the coating consists essentially of (1) sodium carboxymethylcellulose, (2) talc, and (3) sucrose, sodium bicarbonate, or a combination of sucrose and sodium bicarbonate.

20. The protective film coating of claim 19, wherein the coating consists essentially of sodium carboxymethylcellulose, talc, and sucrose.

21. The protective film coating of claim 19, wherein the coating consists essentially of sodium carboxymethylcellulose, talc, and sodium bicarbonate.

22. The protective film coating of claim 17, wherein the coating consists essentially of 50-80% (w/w) sodium carboxymethylcellulose and 15-35% (w/w) talc.

23. The protective film coating of claim 20, wherein the coating consists essentially of 60-70% (w/w) sodium carboxymethylcellulose, 20-30% (w/w) talc, and 5-20% (w/w) sucrose.

24. The protective film coating of claim 21, wherein the coating consists essentially of 60-70% (w/w) sodium carboxymethylcellulose, 20-30% (w/w) talc, and 5-20% (w/w) sodium bicarbonate.

25. A pharmaceutical dosage form, which is a tablet comprising an active pharmaceutical ingredient and a pharmaceutically acceptable carrier, wherein the tablet is coated with a protective film which comprises a polymeric binder and an anti-tackifying agent, wherein the polymeric binder is sodium carboxymethylcellulose and the anti-tackifying agent is talc.

26. The pharmaceutical dosage form of claim 25, wherein the protective film further comprises a dissolution enhancing agent.

27. The pharmaceutical dosage form of claim 26, wherein the dissolution enhancing agent is selected from sucrose and sodium bicarbonate, or a mixture thereof.

28. The pharmaceutical dosage form of claim 25, wherein the active pharmaceutical ingredient is sensitive to decomposition by atmospheric oxygen

29. The pharmaceutical dosage form of claim 28, wherein the active pharmaceutical ingredient is amorphous atorvastatin calcium.
